(19) United States
(12) Plant Patent Application Publication
(10) Pub. No.: US 2013/0305418 P1
(43) Pub. Date: Nov. 14, 2013

(54) Raspberry plant - Evita cultivar

(50) ;

(71) Applicant: SANT'ORSOLA SOCIETA' COOPERATIVA AGRICOLA (Italian Farmers Cooperative), Pergine Valsugana (IT)

(72) Inventors: Marco Giacomelli, Vigolo Vattaro (IT); Alessandra Viliotti, Vigolo Vattaro (IT); Sara Giacomelli, Vigolo Vattaro (IT); Sara Fontanari, Pergine Valsugana (IT)

(21) Appl. No.: 13/986,484

(22) Filed: May 7, 2013

(30) Foreign Application Priority Data
May 14, 2012 (IT) CPVO 20120809

(51) Int. Cl. A01H 5/00

(52) U.S. Cl. PLT/204

(57) ABSTRACT

A new a primocane raspberry cultivar named “Evita” having good fruit colour and flavour, great and firm berries and good tolerance to pests and diseases is disclosed.

### GENUS AND SPECIES

Rubus idaeus L.

### VARIETY DENOMINATION

“Evita”

### BACKGROUND OF THE NEW VARIETY

A new and distinct cultivar of red raspberry (Rubus idaeus L.) hereinafter referred to by the cultivar denomination “Evita” is disclosed. The new variety “Evita” was obtained from the cross by seedling of individual raspberry plants of the varieties Dolomia and Erika.

The crossing for the new cultivar occurred on year 2007 in Vigolo Vattaro (Trento—Italy) and it has been subject to selection on year 2009 and then tested since year 2011. The characteristics of the new cultivar have been found stable and have been transmitted without change through succeeding (more than 5 years) asexual propagations.

Plant breeder's right were applied for with the European Union on May 14, 2012. “Evita” has not been made publicly available.

### SUMMARY OF THE INVENTION

The most outstanding and distinguishing characteristics of the “Evita” cultivar are hereunder reported.
Primocane raspberry plant;
Good fruit colour and flavour;
Great and firm red berries;
Good tolerance to raspberry pests and diseases.

The new cultivar produces two crops per year and the fruits mainly ripen on current season's cane in autumn.

The new cultivar can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar “Evita” is illustrated by the accompanying photographs showing typical specimens of the new variety in colour as true as reasonably possible to make the same colour illustrations of this character by conventional photographic procedures.

FIG. 1 is a view showing both ripe and immature fruits and leaves.

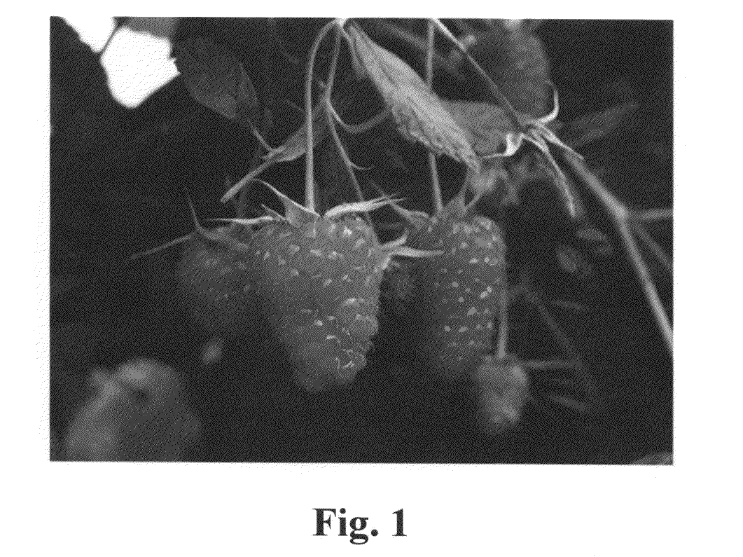

FIG. 2 is a view showing a ripe fruit size and shape and leaves.

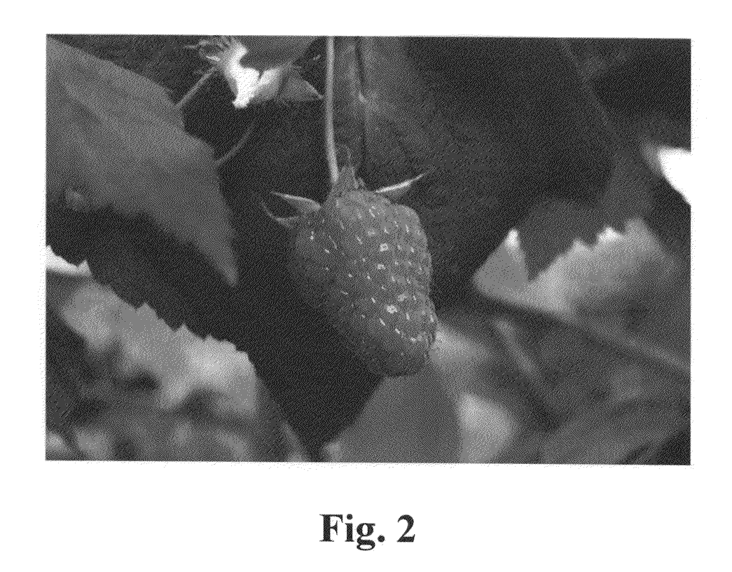

FIG. 3 is a view showing the size, shape and colour at ripening of harvested fruits.

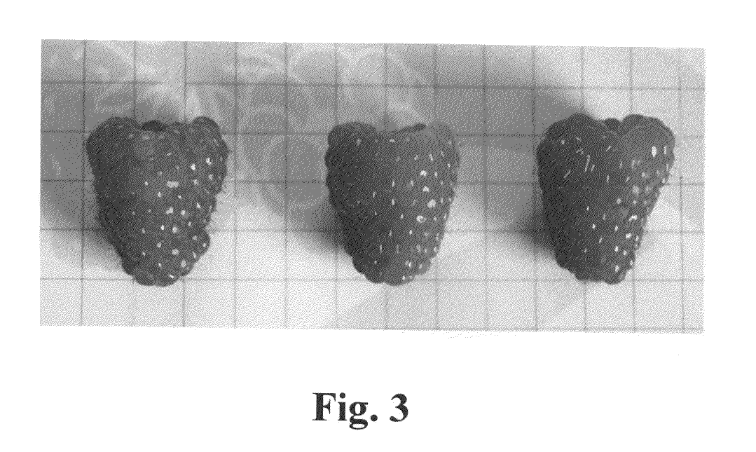

### DETAILED DESCRIPTION

The following detailed description set forth the distinctive characteristics of the new variety “Evita”. The performance of new cultivar for retention of its distinctive characteristics has been evaluated through successive asexual propagations by root cutting in the Applicant's setting in Trentino (Italy) under controlled conditions of temperature (minimal temperature 15° C. and maximal temperature 28° C.) in greenhouse. The growing was the out of soil using peat as substrate.

The plant assessment was performed for 3 years.
Classification:
   Family.—Rosaceae.
   Botanical.—Rubus idaeus L.
   Common name.—Raspberry.
Parentage: Dolomia×Erika.
Plant:
   General habit.—upright canes; strong vigor; very good roots system; adapted to medium winter chill conditions such as those of Northern Italy; high productivity in autumn.
   Number of current season's shoots.—medium.
   Very young shoot.—anthocyanin coloration of apex during rapid growth absent.
   Cane colour.—brown.
   Spines.—present.
   Leaves.—bright green on the superior surface and light green on the inferior surface; concave; odd-pinnate and subdivided in 3-5 ovate and serrate small leafs with medium rugosity.
   Bloom.—on the current season's cane is early to medium and the flowers are typical of raspberry and the blooming is white and begins in medium season.
Fruit:
   Predominant shape.—conical.
   Weight.—6.60 g (approximately).
   Size.—ratio length/width 2.9 cm/2.4 cm (approximately).
   Colour of ripe fruit.—medium red.
   Firmness.—high.
   Main bearing type.—on current year's cane in autumn.
   Storage capacity.—good shelf life.
Pest and disease resistance: “Evita” presents strong canes resistant to pests and diseases; the plants needs few spry treatments for the diseases and pests control.

### Comparison with Parental and Commercial Cultivar

“Evita” differs from the parental varieties in that:
the harvest is anticipated of about 7 days respect to the parent variety Erika;
the crop is about 20% higher than the parent variety Erika;
the fruit at ripening is greater and has a more brilliant colour than the parent variety Dolomia.

The similarities of new cultivar “Evita” with the commercial variety “Polka” are shown in Table 1.

TABLE 1
Comparison between “Evita” and “Polka”

| Characteristic | “Evita” | “Polka” |
|---|---|---|
| fruit color | medium red | dark red |
| fruit size | big | medium |
| Cane color | brown | light-brown |
| plant vigor | strong | medium |

What is claimed:

1. A new and distinct cultivar of raspberry plant as substantially herein shown and described.
